(19) United States
(12) Patent Application Publication
(10) Pub. No.: US 2008/0097233 A1
(43) Pub. Date: Apr. 24, 2008

(54) Method and apparatus for estimating a paco2 value for a patient subject to extra corporeal circulation

(75) Inventors: Tore H. Pedersen, Bekkestua (NO); Frode Kristiansen, Gjettum (NO); Jan Olav Hogetveit, Jar (NO)

Correspondence Address: CHRISTIAN D. ABEL, ONSAGERS AS, POSTBOKS 6963 ST. OLAVS PLASS, NORWAY, N-0130, omitted

(73) Assignee: RIKSHOSPITALET-RADIUMHOSPITALET HF, Oslo (NO)

(21) Appl. No.: 11/568,070

(22) PCT Filed: Apr. 20, 2005
(86) PCT No.: PCT/NO05/00131

§ 371 Date: Dec. 27, 2006

(30) Foreign Application Priority Data
Apr. 20, 2004 (NO) 20041611

(51) Int. Cl. A61B 5/083

(52) U.S. Cl. 600/531

(57) ABSTRACT

The invention relates to a method and an apparatus for estimating a PₐCO₂ value for a patient subject to extracorporeal circulation by means of an oxygenator. The method comprises the steps of measuring a PₑₓCO₂ value in the exhaust gas of the oxygenator and the patient's arterial blood temperature value Tₐ, using a temperature sensor arranged in the oxygenator. The estimated PₐCO₂ value is then calculated, based on the measured PₑₓCO₂ value and the arterial temperature measurement. Advantageously, an average value determined from a predetermined number of recent PₑₓCO₂ values is used in the calculation. The calculation is performed by adding a correction term to the PₑₓCO₂ value, which is composed of a temperature dependent component and an offset component. The correction term may be adjusted by a user. The estimated PₐCO₂ value is presented on a display. A switch provides easy alteration between pH-stat mode and alpha-stat mode.

### TECHNICAL FIELD

The present invention relates in general to the field of medical technology.

More specifically, the invention relates to a method and an apparatus for estimating a PₐCO₂ value representing the arterial partial pressure of CO₂ for a patient subject to extracorporeal circulation by means of an oxygenator.

### BACKGROUND OF THE INVENTION

During cardiopulmonary bypass (CPB) there is a need for reliable, accurate and instant estimates of the arterial blood CO₂ tension (PₐCO₂) of the patient.

Currently, the regular practice for measuring PₐCO₂ during cardiopulmonary bypass involves the manual collection of intermittent blood samples, followed by a separate analysis performed by a gas analyser. This approach involves a substantial delay in the acquiring of data, as well as undesirable manual steps.

Probes for inline blood gas measurements exist, but they are expensive and thus not applicable for routine use.

A well known approach is to measure PₑₓCO₂, i.e. the partial pressure of CO₂ in the exhaust gas output from the oxygenator used in the extracorporeal circulation equipment, and use this measurement as a direct estimate for PₐCO₂.

Changes in temperature inevitably affect the acid-base balance, and management of both pH and CO₂ is of special concern during deep hypothermia. Two commonly used management protocols or blood gas strategies are known as the pH-stat strategy and the alpha-stat strategy. According to the pH-stat strategy, pH is held constant, while temperature and PCO₂ are allowed to change. In alpha-stat strategy, PCO₂ is held constant while the temperature and pH are allowed to change.

When operating according to the pH-stat strategy the direct estimate approach, i.e. using PₑₓCO₂ directly as an estimate for PₐCO₂, is appropriate.

However, when operating according to the alpha-stat strategy, the direct estimate approach leads to seriously inaccurate results, in particular at deep hypothermia.

Several studies have been previously accomplished, relating to the problem of estimating PₐCO₂ values for a patient subject to extracorporeal circulation.

For instance, Potger et al, in the article Membrane Oxygenator Exhaust Capnography for continuously estimating arterial carbone dioxide tension during cardiopulmonary bypass (J. of the American Society of the American Society of Extra-Corporeal Technology, 2003:35:218-223), reports that a correlation has been found between the PₑₓCO₂ and a temperature corrected PₐCO₂.

Likewise, M. J. O'Leary et. al: Oxygenator exhaust capnography as an index of arterial carbon dioxide tension during cardiopulmonary bypass using a membrane oxygenator, (Br. J. of Anaesth 1999 82:(6):843-6), and the subsequent comments by W. M. Weightman et al. (Br. J. Anaesth. 2000 84: 536-537) discuss various relations between PₐCO₂, PₑₓCO₂ and the body temperature of the patient.

However, none of these publications actually disclose a practical, reliable, accurate and cost-effective method and apparatus for estimating PₐCO₂ values for a patient subject to extracorporeal circulation.

### SUMMARY OF THE INVENTION

An object of the present invention is to provide a method and an apparatus for estimating a value (PₐCO₂) representing the arterial partial pressure of CO₂ for a patient subject to extracorporeal circulation by means of an oxygenator, which overcome the disadvantages of the prior art.

A particular object of the present invention is to provide such a method and an apparatus which provide accurate results, which is reliable, easy to operate, and which is inexpensive with regard to manufacturing and use.

A further object of the invention is to provide such a method and an apparatus which may be calibrated before use, and which also may be adjusted by the user during operation.

An additional object of the invention is to provide such a method and an apparatus which utilizes already present devices in the existing extracorporeal circulation equipment, avoiding further components to come in contact with the circulating blood of the patient.

Still another object of the invention is to provide such a method and an apparatus which may be operated according to pH-stat strategy or alpha-stat strategy, whereby the alternation between the two operation modes may be changed easily by the user.

The above objects and further advantages are achieved by a method and an apparatus as set forth in the appended set of claims.

Additional features and principles of the present invention will be recognized from the detailed description below.

It is to be understood that both the foregoing general description and the following detailed description are exemplary and explanatory only and not restrictive of the invention, as claimed.

### BRIEF DESCRIPTION OF THE DRAWINGS

The accompanying drawings illustrate a preferred embodiment of the invention. In the drawings,

FIG. 1 is a schematic block diagram illustrating an apparatus according to the invention included in its operating environment, and

FIG. 2 is a schematic flow chart illustrating a method according to the invention.

### DETAILED DESCRIPTION OF THE INVENTION

Reference will now be made in detail to the present invention, examples of which are illustrated in the accompanying drawings. Wherever possible, the same reference numbers will be used throughout the drawings to refer to the same or like parts.

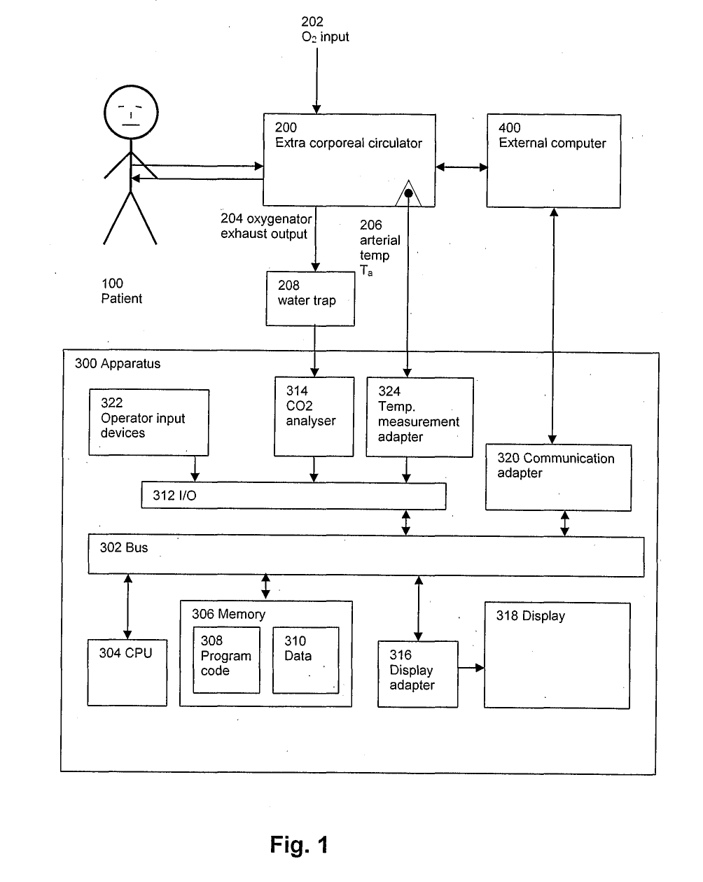

FIG. 1 is a schematic block diagram illustrating an apparatus according to the invention included in its operating environment.

The illustrated apparatus 300 is an apparatus for estimating a PₐCO₂ value, representing the arterial partial pressure of CO₂ for a patient 100 subject to extracorporeal circulation by means of an oxygenator incorporated in the extracorporeal circulator 200.

The apparatus 300 comprises a measuring device 314, in particular a CO₂ analyzer, for measuring a PₑₓCO₂—value representing the partial pressure of CO₂ in the substantially continuous and laminar flow of exhaust gas which is supplied by the oxygenator exhaust output 204.

The gas inlet of the CO₂ analyzer 314 is equipped with a connector adapted for the releasable connection of a disposable tube, which is equipped with a disposable water trap 208, conveying exhaust gas from the oxygenator exhaust output 204.

The CO₂ analyzer 314 comprises a CO₂ sensor, which may be, e.g., based on measurements of the absorption of infrared radiation through a sample cell which contains the gas to be analyzed. The CO₂ analyzer also comprises electronic circuitry for signal processing. An example of a CO₂ analyzer applicable for use with the present invention is the Square One 2125 gas analyzer. A disclosure of an appropriate CO₂ analyzer is also presented in U.S. Pat. No. 5,932,877, which is hereby incorporated by reference.

The apparatus is designed as a processor-based instrument with a regular processor bus structure. As illustrated, a bus 302 is connected to a processing device 304, in particular a microprocessor. The bus 302 is further connected to a memory 306, comprising a program code portion 308, preferably contained in a non-volatile part of the memory 306 such as an EEPROM or a Flash memory, and a data portion 310, preferably contained in a volatile part of the memory 306 such as a RAM. Advantageously, non-executable data, such as parameters that should be maintained even if the power supply of the apparatus is switched off, may also be held in the non-volatile part of the memory 306.

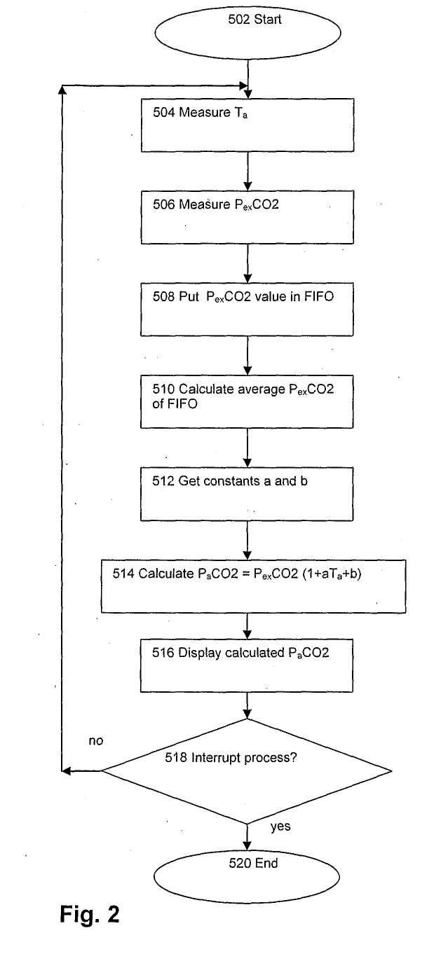

The processing device 304 is arranged to execute the program code held in the program code portion 308 in the memory 306. In particular, the program code comprises instructions which cause the processor device 304 to perform the steps of the method according to the invention or to perform actions which in turn causes the apparatus 300 to perform the steps of the method according to the invention, as described below in particular with reference to FIG. 2. The coding of an appropriate program code implies an ordinary task for a person skilled in the art, based on the disclosure set forth in the present specification.

The processor device 304 is thus arranged to, when executing the program code 308, to calculate the estimated PₐCO₂ value, dependent on the PₑₓCO₂ value measured in said exhaust gas, and a the temperature value Tₐ of the patient's arterial blood temperature.

The apparatus advantageously comprises a low pass filter, arranged for low pass filtering said PₑₓCO₂ value provided by the CO₂ analyzer 314. The aim of such a filter is to reduce the effects of rapid fluctuations in the measured PₑₓCO₂ value. This filter may be implemented in several ways. For instance, it may be implemented as an analog LP filter, reducing the most rapid changes in an analog signal corresponding to the PₑₓCO₂ value.

More advantageously, the LP filter is implemented digitally, as the processing device 304 is arranged to calculate an average value of a number of recently measured PₑₓCO₂ values. This may again be implemented by putting each incoming PₑₓCO₂ value in a FIFO queue structure with a predetermined number N of elements, and by arranging the processing device to read all elements in the FIFO data structure, and to calculate a running average value for the recent N PₑₓCO₂ values. The number N may be selected by the skilled person, based on, inter alia, the sampling rate of the PₑₓCO₂ readings. In an example embodiment, N=20.

The processing device 304 is further arranged to perform the calculating of the estimated PₐCO₂ value by adding a correction value to the PₑₓCO₂ value of partial pressure in the exhaust gas.

The correction value is advantageously substantially linearly dependent on said temperature value Tₐ. In particular, the correction value is calculated as a proportionality constant (a), multiplied by the temperature value Tₐ 206, added to an offset constant (b).

The proportionality constant (a) and the offset constant (b) are advantageously generated in advance, by means of a preceding calibration procedure. The constants are then stored in a part of the memory 306, preferably in a non-volatile part of the memory 306.

The apparatus 300 further comprises input devices 322 for operation by a user of the apparatus. In particular, the apparatus 300 comprises an input device which is arranged for setting or adjusting the proportionality constant (a) and the offset constant (b). This input device may be embodied as analog operating devices such as rotating buttons, or by pushbuttons such as the keys of a keyboard, whereby the user may, e.g., enter numerical values.

The input devices 322, the CO₂ analyzer 314 and a temperature measurement adapter 324 are all connected to an I/O device 312, which in turn is connected to the internal bus 302 of the apparatus 300.

The arterial temperature Ta is measured by means of a temperature sensor 206, in particular a thermocouple, which is inserted in the oxygenator in the extracorporeal circulator 200, and thereby arranged to measure the arterial blood temperature.

The temperature sensor 206 is connected to the input of the temperature measurement adapter 324, which is arranged to convert the sensor signal to a digital signal, adapted to be read by the system bus 312.

The apparatus 300 advantageously comprises a display adapter 316, connected to the bus 302 and further to a display 318. The display 318 may e.g. be an LCD display.

The processing device 304 is further arranged to display the estimated PₐCO₂ value on the display 318.

The input devices 322 advantageously further comprise a mode switch (not illustrated). The operation of the mode switch provides easy alteration between pH-stat mode and alpha-stat mode. The processing device is arranged to, when the mode switch is in pH-stat position, to present the uncorrected PₑₓCO₂ value on the display. The processing device is further arranged to, when the mode switch is in the alpha-stat position, to present the estimated (corrected) PₐCO₂ value on the display.

Alternatively, the processing device may be arranged to set both constants a, b equal to zero when the mode switch is determined to be in ph-stat position.

The input devices 322 may further comprise a start button (not illustrated). The operation of the start button initiates the estimating process according to the invention.

The processing device is advantageously arranged to reiterate the steps of calculating estimated PₐCO₂ values, each time using updated measurement values. This results in a quasi-continuous process, repeatedly providing estimated PₐCO₂ values.

The input devices 322 also advantageously comprise a stop button (not illustrated). The processing device is arranged to terminate the reiteration of the estimating process upon the operation of the stop button.

A communication adapter 320 is connected to the bus 302, enabling communication between the apparatus 300 and an operatively connected, external computer 400. The computer 400 may advantageously also be operatively connected to the extracorporeal circulating equipment 200.

Although not illustrated, the apparatus also comprises a power supply, a casing, connectors for gas and electric signals, operating elements et cetera. The selection and arrangement of such regular components, as well as the constructional details leading to a complete, working apparatus, may readily be performed by a person skilled in the art, without inventive efforts. The skilled person will further realize that the practical implementation of the apparatus should aim at complying with the IEC 60601-1 standard, published by the International Electrotechnical Commission (IEC).

FIG. 2 is a schematic flow chart illustrating a method according to the invention.

The method illustrated in FIG. 2 is preferably performed as a microprocessor-implemented process, advantageously by the processor device 304 illustrated in FIG. 1.

The illustrated method is a method for estimating a PₐCO₂ value representing the arterial partial pressure of CO₂ for a patient subject to extracorporeal circulation by means of an oxygenator in an extracorporeal circulator.

The method starts at the initiating step 502, e.g. initiated by the operation of a start button included in the operator input devices 322 illustrated in FIG. 1.

Next, in step 504, an arterial blood temperature measurement Tₐ is provided. Advantageously, this value is acquired by use of a temperature sensor usually incorporated in the extracorporeal circulating equipment 200.

Next, in step 506, a measurement PₑₓCO₂ of the partial pressure of CO₂ in the exhaust gas output 204 from the oxygenator is provided.

It will be apparent to the skilled person that the measurement steps 504 and 506 may be performed in the opposite order, or even simultaneously or concurrently, if so desired.

Advantageously, the PₑₓCO₂ value is low pass filtered. This is accomplished by the filtering steps 508 and 510. In step 508, the presently acquired PₑₓCO₂ value is put in a FIFO queue data structure of e.g. N=20 elements. Then, in step 510, the average or mean value of all the FIFO elements are calculated. This leads to a LP filtered PₑₓCO₂ value identical to the mean value of the last N=20 acquired instant values. This filtering reduces the effects of rapid fluctuations in the instant PₑₓCO₂ values.

Of course, the filtering may be omitted or replaced by an alternative filtering process. As an example, another calculating result than the plain arithmetic mean value may be derived from the FIFO values. The resulting output may rather be a weighted average, wherein the most recent instant PₑₓCO₂ values are weighted more dominantly than the less recent instant values. Other possibilities exist within the apprehension of the skilled person.

In step 512 a proportionality constant (a) and an offset constant (b) are provided, preferably fetched from a memory 306. The constants may be pre-generated by a preceding calibration procedure and then stored in the memory. The constants may further advantageously be set or adjusted by an input device included in the operator input devices 322, as explained previously with reference to FIG. 1.

The calibration procedure for pre-generating the constants a and b may be performed by the following method:

For a number of different arterial blood temperatures Tₐ (e.g., 32° C., 35° C. and 38 °C.), and preferably also for a number of different patients (e.g., 10 patients), the PₑₓCO₂ values measured by the CO₂ analyzer is measured and recorded. For each arterial blood temperature value Tₐ the corresponding “true” PₐCO₂ value is also recorded, provided by manual collection of a blood sample followed by a separate analysis performed by a reference bloodgas analyzer. This leads to a multiple data set of corresponding values for temperatures, PₑₓCO₂ and PₐCO₂. The values for the constants a and b are then easily calculated by regular linear regression, thus establishing the set of constant values (a, b) that most properly fits the equation PₐCO₂=PₑₓCO₂+aTₐ+b.

Calibration values of the constants a and b maybe recorded and supplied for downloading into a memory as constant values associated with a specific oxygenator.

Subsequent to step 512 of providing pre-generated constants a and b, the main calculating step 514 is performed.

In this step, the estimated PₐCO₂ value is established by the formula

PₐCO₂=PₑₓCO₂ +aTₐ+b,

wherein PₑₓCO₂ is the preferably averaged or filtered PₑₓCO₂ measurement value, Tₐ is the measured arterial blood temperature and a, b are constants as explained above.

That is, the estimated PₐCO₂ is calculated as the PₑₓCO₂ value, corrected by adding to it a correction term (aTₐ+b).

As can be seen of the formula, the correction value is preferably linearly dependent on the arterial blood temperature value (Tₐ).

However, the skilled person will realize that other models for providing a correction value exist, including correction values provided as 2^{nd}, 3^{rd} or even higher order polynomials of the arterial temperature value. In such cases, a corresponding number of constants are evidently necessary. Tests have however shown that a linear approximation is appropriate for practical purposes.

Next, in step 516, the estimated PₐCO₂ is presented on a display.

If the process is to be terminated, e.g. activated by a pressed stop-button included in the operator input devices 322, the terminating step 520 is performed. Else, the process is reiterated by returning to step 504.

Several modifications and adaptations of the present invention will be apparent to those skilled in the art from consideration of the specification and practice of the invention disclosed herein.

For instance, although the apparatus is illustrated as a complete instrument wherein the processing device, the display and operating devices are all included, the skilled person will readily realize that alternative embodiments also exist within the scope of the invention as defined in the claims. The apparatus may thus be, e.g., embodied as a virtual instrument operating on a general purpose computer such as a PC, executing virtual instrument software such as e.g. LabView, which is further configured in order to operate according to the present invention. LabView is a computer software product for a data acquisition and virtual instrumentation, manufactured by National Instruments Corp., which is well-known to a person skilled in the art. In this case, the computer will either include or operatively be connected to peripheral devices necessary to practice the invention, in particular the CO₂ analyzer and an input port for the arterial temperature signal Tₐ. However, functions such as the operating devices or switches may effortlessly be implemented as software modules, operating in conjunction with regular computer input devices such as a keyboard and a mouse. The display 31S may correspondingly be substituted by a regular computer display.

The above detailed description of the invention has been presented for purpose of illustration. It is not exhaustive and does not limit the invention to the precise form disclosed. Modifications and variations are possible in light of the above teachings or may be acquired from the practicing of the invention.

1. Method for estimating a value (PₐCO₂) representing the arterial partial pressure of CO₂ for a patient subject to extracorporeal circulation by means of an oxygenator, comprising the steps of:
measuring a value (PₑₓCO₂) representing the partial pressure of CO₂ in the exhaust gas of the oxygenator,
measuring a temperature value (Ta) representing the patient's arterial blood temperature, and
calculating the estimated value (PₐCO₂) representing the arterial partial pressure of CO₂, dependent on said value (PₑₓCO₂) representing said partial pressure of CO₂ in said exhaust gas and said temperature value (Tₐ).

2. Method according to claim 1,
wherein said value (PₑₓCO₂) representing the partial pressure of CO₂ in said exhaust gas is low pass filtered, reducing the effects of rapid fluctuations in the measured value.

3. Method according to claim 1 or 2, wherein said value (PₑₓCO₂) is calculated as an average value of a number of recently measured values (PₑₓCO₂) of partial pressure in said exhaust gas.

4. Method according to one of the claims 1-3, wherein said step of calculating said estimated value (PₐCO₂) comprises
adding a correction value to the value (PₑₓCO₂) of partial pressure in said exhaust gas, said correction value being substantially linearly dependent on said temperature value (Tₐ).

5. Method according to claim 4,
wherein said correction value is calculated as a proportionality constant (a) multiplied by the temperature value (Tₐ) added to an offset constant (b).

6. Method according to claim 5,
wherein said proportionality constant (a) and said offset constant (b) are pre-generated by a calibration procedure and stored in a memory.

7. Method according to claim 5 or 6,
wherein said proportionality constant (a) and said offset constant (b) may be set or adjusted by an input device.

8. Method according to one of the claims 1-7,
wherein said step of measuring the temperature value (Tₐ) is performed by means of a temperature sensor arranged in an extracorporeal circuit which comprises said oxygenator.

9. Method according to one of the claims 1-8,
wherein said step of measuring the value (PₑₓCO₂) representing the partial pressure of CO₂ in the exhaust gas of the oxygenator comprises exposing a substantially continuous and laminar flow provided by the oxygenator exhaust output to the input of a CO₂ sensor.

10. Method according to one of the claims 1-9, further comprising the step of
displaying the estimated value (PₐCO₂) presenting the arterial partial pressure of CO₂ on a display.

11. Method according to one of the claims 1-10, reiterated as a pseudo-continuous process until the receipt of a stop signal.

12. Apparatus for estimating a value (PₐCO₂) representing the arterial partial pressure of CO₂ for a patient subject to extracorporeal circulation by means of an oxygenator, comprising
a measuring device for measuring a value (PₑₓCO₂) representing the partial pressure of CO₂ in the exhaust gas of the oxygenator,
a temperature measuring device for providing a temperature value (Tₐ) representing the patient's arterial blood temperature, and
a processing device, arranged to perform the step of
calculating the estimated value (PₐCO₂) representing the arterial partial pressure of CO₂, dependent on said value (PₑₓCO₂) representing said partial pressure of CO₂ in said exhaust gas and said temperature value (Tₐ) representing the patient's arterial blood temperature.

13. Apparatus according to claim 12, further comprising a low pass filter, low pass filtering said value (PₑₓCO₂) representing the partial pressure of CO₂ in said exhaust gas, reducing the effects of rapid fluctuations in the measured value.

14. Apparatus according to claim 12 or 13, wherein said processing device is arranged to calculate the value (PₑₓCO₂) as an average value of a number of recently measured values (PₑₓCO₂) of partial pressure in said exhaust gas.

15. Apparatus according to one of the claims 12-14, wherein said processing device is arranged to perform said step of calculating said estimated value (PₐCO₂) by adding a correction value to the value (PₑₓCO₂) of partial pressure in said exhaust gas, said correction value being substantially linearly dependent on said temperature value (Tₐ).

16. Apparatus according to claim 15,
wherein said processing device is arranged to calculate said correction value as a proportionality constant (a) multiplied by the temperature value (Tₐ) added to an offset constant (b).

17. Apparatus according to claim 16,
wherein said proportionality constant (a) and said offset constant (b) are pre-generated by a calibration procedure and stored in a memory.

18. Apparatus according to claim 16 or 17, further comprising an input device,
wherein said processing device is arranged for setting or adjusting said proportionality constant (a) and said offset constant (b) by means of the input device.

19. Apparatus according to one of the claims 12-18,
wherein said temperature value (Ta) is measured by means of a temperature sensor arranged in an extracorporeal circuit which comprises said oxygenator.

20. Apparatus according to one of the claims 12-19,
wherein said measuring device for measuring the value (PₑₓCO₂) representing the partial pressure of CO₂in the exhaust gas of the oxygenator comprises a CO₂ sensor arranged in conduit supplied by a substantially continuous and laminar flow provided by the oxygenator exhaust output.

21. Apparatus according to one of the claims 12-20, further comprising a display, wherein said processing unit is further arranged to display the estimated value (PₐCO₂) presenting the arterial partial pressure of CO₂ on said display.

22. Apparatus according to one of the claims 12-21, further comprising a stop signal operating device, wherein said processing device is arranged to reiterate said calculating step, using updated measurement values, until the receipt of a stop signal from the stop signal operating device.
